(19) United States
(12) Patent Application Publication
(10) Pub. No.: US 2021/0268031 A1
(43) Pub. Date: Sep. 2, 2021

(54) Cartilage Regeneration by Synovial Fluid-Derived Stem Cells and Their Derivatives

(71) Applicant: Tarachon LLC, Brooklyn, NY (US)

(72) Inventors: Tara Sharif, Brooklyn, NY (US); Vijay Vad, Brooklyn, NY (US)

(73) Assignee: Tarachon LLC, Brooklyn, NY (US)

(21) Appl. No.: 17/201,659

(22) Filed: Mar. 15, 2021

Related U.S. Application Data
(63) Continuation-in-part of application No. PCT/US2019/052081, filed on Sep. 20, 2019.
(60) Provisional application No. 62/734,710, filed on Sep. 21, 2018.

(51) Int. Cl. A61K 35/32; C12N 5/0775; C12N 5/077

(52) U.S. Cl. CPC A61K 35/32; C12N 5/0655; C12N 5/0668

(57) ABSTRACT

A method of making mammalian chondroblast cells or progenitors thereof and a method for their use. A method of making exosomes and signaling factors and a method for their use.

### TECHNICAL FIELD

This disclosure relates to the field of regenerative medicine. Specifically, this disclosure relates to cartilage regeneration by Synovial Fluid-Derived Stem Cells (SF-MSCs) and exosomes, or growth/differentiating factors excreted by mesenchyme stem cells.

### BACKGROUND

Osteoarthritis (OA) is the most common faun of arthritis. It develops when the protective hyaline cartilage on the ends of the bones wear down in time. Repetitive movements, heavy lifting, weakness of muscles associated with the joints and athletic injuries can also lead to cartilage breakdown and OA. Osteoarthritis causes pain; inflammation, and reduced motion in all joints, but mostly in the joints of the knees, hips, shoulders, hands and spine.

An estimated 10%-15% of all adults over the age of 60 will develop some degree of OA (over 30 million adults in the US), with prevalence being higher among women, and on the rise due to the ageing of the populations and obesity. OA is also a source of morbidity and economic loss in the racehorse and companion animal populations.

Aside from osteoarthritis, a large population of younger subjects are afflicted with injuries to cartilage or ligaments of the joints. Common acute injuries of the joint involve damage to anterior and posterior cruciate ligament (ACL and PCL), medial and lateral collateral ligaments (MCL and LCL), and menisci.

Although pain, reduced mobility and other symptoms of damaged joints can be temporarily managed by routine modalities (pain killers, injection of steroids, hyaluronic acid in the joints, etc.), the underlying cause of the disorder persists. Advanced cell therapy treatments have sought to regenerate the damaged cartilage and other components of the joints and restore the normal joint functions. Cells commonly used in regeneration include autologous mesenchyme stem cells derived from bone marrow, adipose tissue and full grown cartilage. Issues associated with the use of these sources include limited collection site, limited number of relevant stem cells, and fully differentiated cells with low regenerative capacity. Heterologous sources are also used for stem cell therapy, and include umbilical, embryonic or placental tissues. These modalities involve complicated harvesting processes, possible immunological reactions, and sub-optimal number of compatible cells.

### SUMMARY

This disclosure provides a method of making mammalian chondroblasts, or progenitors thereof, comprising: extracting free-floating cells from synovial cavity of a subject; sorting the free-floating cells for categorization purposes, into at least two populations of cells:

(i) Synovial Fluid-Derived “Mesenchyme Stem” Cells (SF-MSCs)/“Progenitor” Cells (SF-PCs), positive for CD90, CD73 and CD105 cell surface markers;
(ii) Non-stem cells, which are synovial membrane cells, endothelial cells, mature chondrocytes, macrophages and other non-stem cells; Growing said synovial fluid-derived free-floating cells without separating different cell types in vitro, colonizing SF-MSCs/SF-PCs and differentiating them to chondroblasts, or progenitors thereof, without the use of any growth factor or differentiating agents; and resulting in an almost homogeneous (for example, about 88% or more) population of chondroblasts, or progenitors thereof, in 2-3 weeks in vitro.

In other aspects, this disclosure provides a method of treating a subject comprising: administering a therapeutically effective amount of the chondroblasts, or progenitors thereof, made by a disclosed method to a subject in need thereof.

In other aspects, this disclosure provides a method of making exosomes and/or other growth/differentiating factors (collectively, “signaling factors”), comprising: collecting exosomes, and/or other growth/differentiating factors, excreted by:

(i) synovial fluid mesenchyme stem cells or progenitor cells derived from injured or healthy joint tissues, or
(ii) mesenchyme stem cells or progenitor cells isolated from a source other than synovial fluid, wherein said source is adipose tissue or bone marrow from a mammalian subject, including a young subject; or an embryo, a fetus (including umbilical cord and placenta) or a mixture of these sources, after culturing said mesenchyme stem cells or progenitor cells for at least two weeks in vitro.

In other aspects, this disclosure provides a method of differentiating allogeneic mesenchyme stem cells to chondroblast, or progenitors thereof, comprising: culturing the allogeneic mesenchyme stem cells in close proximity to the exosomes or other growth/differentiating factors made by a disclosed method.

In other aspects, this disclosure provides a method of treating a subject comprising: administering a therapeutically effective amount of the exosomes, or other growth/differentiating factors, made by a disclosed method to a subject in need thereof.

In other aspects, this disclosure provides a method of treating a subject comprising: administering to a subject/patient in need thereof a therapeutically effective amount of the exosomes or other growth/differentiating factors made by a disclosed method, exosomes or other growth/differentiating factors made by embryonic mesenchyme stem cells, by a combination of exosomes or other growth/differentiating factors from both adult and embryonic sources, or by exosomes or other growth/differentiating factors made by mesenchyme stem cells originated from sources other than synovial fluid including adipose tissue and bone marrow; and fetal, umbilical and placental tissues.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1A-FIG. 1L show CD analysis of chondroblast cells made by a method of this disclosure.

FIG. 1A-FIG. 1C: day 0; FIG. 1D-FIG. 1F: day 16. FIG. 1A—CD73: 16.21%; FIG. 1B—CD90: 6.02%; FIG. 1C—CD105: 17.39%. FIG. 1D—CD73: 86.60%; FIG. 1E—CD90: 95.7%; FIG. 1F—CD105: 99.98%.

FIG. 1G-FIG. 1I: double positions, day 0; FIG. 1J-FIG. 1L: double positions, day 16. FIG. 1G—CD90-CD105: 1.36%; FIG. 1H—CD105-CD73: 4.19%; FIG. 1I—CD90-CD73: 1.83%, and triple marker: 2.46%.

FIG. 1J—CD90-CD105: 97.72%; FIG. 1K—CD105-CD73: 84.13%; FIG. 1L—CD90-CD73: 85.25%, and triple marker: 89.03%.

FIG. 1M and FIG. 1N—PAS staining showing presence of cartilage components. The chondroblast cells were made from a 27-year old male patient with medial femoral condyle grade IV knee injury.

FIG. 2A—FIG. 2O show another example of CD analysis and PAS staining of chondroblast cells made by a method of this disclosure.

FIG. 2A-FIG. 2C: day 0; FIG. 2D-FIG. 2F: day 15.

FIG. 2A—CD73: 1.81%; FIG. 2B—CD90: 7.13%; FIG. 2C—CD105:33.04%. FIG. 2D—CD73: 98.9%; FIG. 2E—CD90: 95.54%; FIG. 2F—CD105: 47.53%.

FIG. 2G-FIG. 2I: double positions, day 0; FIG. 2J-FIG. 2L: double positions, day 15. FIG. 2G—CD90-CD105: 3.2%; FIG. 2H—CD105-CD73: 1.4%; FIG. 2I—CD90-CD73: 0.37%, and triple marker: 1.65%. FIG. 2J—CD90-CD105: 45.5%; FIG. 2K—CD105-CD73: 37.92%; FIG. 2L—CD90-CD73: 95.74%, and triple marker: 59.73%.

FIG. 2M, FIG. 2N and FIG. 2O—PAS staining showing presence of cartilage components. The cells were made from a 22 year old male patient with ACL injury and meniscus lesion.

FIG. 3A-FIG. 3N show CD analysis and PAS staining of chondroblast cells made by a method of this disclosure, derived from the healthy knee and injured knees of the same patient. FIG. 3A-3C: day 0, Injured Knee; FIG. 3D-FIG. 3F: day 22, Injured Knee. FIG. 3A—CD73: 2.06%; FIG. 3B—CD90: 1.71%; FIG. 3C—CD105: 22.14%. FIG. 3D—CD73: 92.21%; FIG. 3E—CD90: 99.3%; FIG. 3F—CD105: 97.16%. FIG. 3G-3I: day 0, Healthy Knee; FIG. 3J-FIG. 3L: day 22, Healthy Knee. FIG. 3G—CD73: 2.93%; FIG. 3H—CD90: 0.42%; FIG. 3I—CD105: 25.9%. FIG. 3J—CD73: 98.94%; FIG. 3K—CD90: 90.94%; FIG. 3L—CD105: 63.24%.

FIG. 3M shows PAS staining of SF-MSC made from the healthy knee joint; FIG. 3N shows PAS staining of cartilage components from the culture of the injured knee of the same patient. Although flow cytometry profile of SF-PCs derived from healthy and injured knees were not statistically different, PAS staining showed higher presence of cartilaginous nodules in the culture of SF-PCs derived from the injured knee. The cells were made from a 32 year old female patient with chondral and patellar lesions grade IV.

FIG. 4A-FIG. 4E Show implantation of chondroblast-seeded scaffold to the site of a focal cartilage injury. The cells were made from a 29 year old male patient with 1×1.5 cm grade III focal medial femoral condyle lesion. FIG. 4A: damaged area; FIG. 4B: excised damaged cartilage; FIG. 4C: immediately after implantation; FIG. 4D: 5 weeks post-op MM indicating adherence of the implant; FIG. 4E: 1 year post-op MM indicating healing and cartilage regeneration.

FIG. 5 shows Chondroblast/Progenitor cell-derived exosomes from the Passage 2 culture of a female subject measured on Malvern Nanosight machine. Analysis measured 2.8×10¹⁰/ml exosomes with average diameter of 80.2 nm.

FIG. 6A-FIG. 6E Show induction of formation of Collagen type II by Chondroblast/Progenitor cells of an older patient, treated with exosomes isolated from a Chondroblast/Progenitor cells culture supernatant of a younger patient. The older patient is a 77 year old patient with osteoarthritis who underwent total knee replacement. FIG. 6A and FIG. 6B: Group treated with autologous serum—Phase contrast microscopy (FIG. 6A) and Immunohistochemistry with Collagen Type II, 100× (FIG. 6B). No collagen type II was detected. FIG. 6C-FIG. 6E: Group treated with supernatant/exosomes from a younger patient: Phase contrast microscopy (FIG. 6C) and Immunohistochemistry with Collagen Type II, 100× (FIG. 6D) and 200× (FIG. 6E). Collagen type II formation was demonstrated in the cytoplasm (whitish cloud at 100× and whitish cytoplasmic content at 200×).

FIG. 7 indicates significantly higher number of SF-MSCs derived from male versus female subjects

FIG. 8 shows a progressive decline in the number of harvested SF-MSCs in synovial fluid of patients from the age 18 to age 79.

FIG. 9 shows “colonization” of SF-MSCs/SF-PCs after 18 days in culture.

### DETAILED DESCRIPTION

As used herein, the word “a” or “plurality” before a noun represents one or more of the particular noun. For example, the phrase “a mammalian cell” represents “one or more mammalian cells.”

As used herein, the terms “subject” and “patient” are used interchangeably. A patient or a subject can be, for example and without limitation, a human subject, a racehorse or other mammals such as a companion animal (for example, a dog, a cat, etc.). A subject is any mammal that may benefit from the disclosed methods and compositions.

As used herein, the term “Progenitor” cell refers to a stem cell that is in a further stage of cell differentiation. Progenitor cells are unipotent or oligopotent and can get activated in response to injury and other cues, to initiate repair.

As used herein, the terms “chondroblast” refers to “Progenitor” cells that are partially or fully differentiated, and in essence, these two terms are used interchangeably. When positioned in the right milieu, chondroblasts will form chondrocytes. A chondroblast is a chondrocyte at an earlier stage of growth and development.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

The term “effective amount” or “a therapeutically effective amount” refers to an amount of an agent that provides a beneficial effect to a patient. The term “effective amount” or “a therapeutically effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease or disorder in a patient, or any other desired alteration of a biological system. A beneficial effect can take the form of an improvement over baseline, i.e., an improvement over a measurement or observation made prior to initiation of therapy according to the method. An effective amount or a therapeutically effective amount can be administered in one or more administrations.

The term “autologous” refers to the use of the stem cells, harvested from the same subject who receives it.

The term “allogeneic” refers to the use of the stem cells when the donor is different from the recipient.

The term “anterior cruciate ligament” (ACL) refers to the ligament that attaches the front of the bone of the lower leg, tibia, to the back of the bone of the thigh, femur, in each knee.

The term “posterior cruciate ligament” (PCL) also refers to the ligament that connects tibia with femur, but runs behind the ACL.

The term “medial collateral ligament” (MCL) refers to the ligament that attaches the medial lower tip of the femur to the medial upper tip of the tibia

The term “lateral collateral ligament” (LCL) refers to the ligament located on the outside of the knee joint, connecting the bottom of femur to the top of the smaller lower leg bone, fibula.

The term“meniscus” refers to a c-shaped cartilage pad located in the knee joint between femur and tibia.

The term “hyaluronic acid” refers to a major component of the extracellular matrix. It is a non-sulfated glycosaminoglycan molecule located in several tissues including the connective tissue and the articular cartilage. It provides lubrication in the joints and also contributes to cell proliferation and migration.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Osteoarthritis (OA) is the most common form of arthritis and is developed when the protective hyaline cartilage on the ends of the bones wear down in time. Repetitive movements, heavy lifting, weakness of muscles associated with the joints and athletic injuries can also lead to cartilage breakdown and OA. Osteoarthritis causes pain, inflammation, and reduced motion in all joints, but mostly in the joints of the knees, hips, shoulders, hands and spine. An estimated 10%-15% of all adults aged over 60 will develop some degree of OA (over 30 million adults in the US), with prevalence being higher among women, and on the rise due to the ageing of the populations and obesity. OA is also a source of morbidity and economic loss in the racehorse and companion animal populations.

Aside from OA, a large population of younger subjects are afflicted with injuries to cartilage or ligaments of the joints. Common acute injuries of the joint involve damage to anterior and posterior cruciate ligament (ACL and PCL), medial and lateral collateral ligaments (MCL and LCL), Patellar cartilage and menisci.

Although pain, reduced mobility and other symptoms of damaged joints can be temporarily managed by routine modalities (pain killers, injection of steroids or hyaluronic acid in the joints, etc.), the underlying cause of the disease persists. Advanced cell therapy treatments have sought to replace the damaged cartilage and other components of the joints and restore the normal joint functions. Cells commonly used in regeneration include autologous mesenchyme stem cells derived from bone marrow, adipose tissue and full grown cartilage. Issues associated with the use of these sources include limited collection site and fully differentiated cells with low regenerative capacity. Also used for stem cell therapy are heterologous sources including umbilical, embryonic or placental tissues. These modalities involve complicated harvesting processes, possible immunological responses, and fewer than optimal number of compatible cells.

Synovial fluid contains free floating mesenchymal stem cells that are developmentally more closely related to chondrocytes and therefore are better suited to regenerate cartilage in comparison to other mesenchyme stem cells. Further, synovial fluid contains several hundred fold more chondrogenic mesenchyme stem cells than bone marrow or adipose tissue. Synovial fluid is readily attainable, requires no invasive surgical procedures and has a great capacity to demonstrate chondrogenesis, as shown herein.

This disclosure provides a method of making mammalian chondroblasts, or progenitors thereof, comprising: extracting free-floating cells from synovial cavity of a subject; sorting the free-floating cells for categorization purposes, into at least two populations of cells:

(i) synovial fluid-derived mesenchyme “Stem” cells (SF-MSCs)/“Progenitor” cells (SF-PCs), positive for CD90, CD73 and CD105 cell surface markers,
(ii) and Non-stem cells, which are synovial membrane cells, endothelial cells, mature chondrocytes, macrophages and other non-stem cells; Growing said synovial fluid derived free-floating cells without separating different cell types in vitro, colonizing the population of cells comprising SF-MSCs/SF-PCs and differentiating the cells to chondroblasts, or progenitors thereof, without the use of any growth factor or differentiating agents; and resulting in an almost homogeneous (in some embodiments, about 88%, 90%, 95%, or more) population of chondroblasts, or progenitors thereof, in 2-3 weeks in vitro.

In certain embodiments, the method further comprises resulting in the formation of cartilage nodules/components in vitro and/or in vivo. In some embodiments, the synovial cavity is from the injured subjects. In some embodiments, the synovial fluid is extracted from injured synovial cavity of the subject. In some embodiments, the synovial fluid is extracted from healthy synovial cavity of the subject. In some embodiments, the subject is human, racehorse, or a companion animal. The subject may be any animal.

Exemplary Method of Extraction of Synovial Fluid: The procedure is performed in sterile conditions. The knee of a subject is cleaned with iodine solution from the middle of the upper to the mid lower leg. Sterile surgical drapes are used to cover the knee. The entry point of arthrocentesis is marked between patella and femoral condyle, at half an inch proximal and half an inch lateral to the patella. A 27-gauge, 0.4 mm in diameter needle and a 20-ml syringe are used to inject 20 ml of physiological solution into the knee joint. To mobilize the free floating synovial fluid cells, the joint is gently flexed and extended a few times. The combination of the synovial fluid and physiological solution is then aspirated and harvested. The aspirate is often an amber color solution, but at times it can include small amounts of blood. The cell count and cytometry instruments exclude the red and white blood cells. Cell viability is determined by 0.4% trypan blue exclusion. Using this procedure, on average, 1.27 million SF-MSC/SF-PC cells with 85-98% viability comprising between 0.5-3.0% of the total cell population are obtained from each harvest.

The harvesting of synovial fluid can also be done concurrent with portal arthroscopy, should the subject need to have this procedure for other reasons. Using this technique, saline is injected at the point of entry of the scope, and the drainage is done using a collecting tube.

Other harvesting methods can be used.

Exemplary Method of Sorting of the Synovial Fluid-Derived Cells: A suspension of 1×10⁵ synovial fluid cells is placed in each of four 1 ml polystyrene tubes. To 3 tubes, 10 μl solution of: monoclonal antibodies to CD90, CD73 or CD105 are added. The 4th tube is used for control. The tubes are incubated for 30 minutes at 4° C. The data is obtained in a BD (Becton-Dickinson) FACSCalibur flow cytometer and analyzed by CellQuest™ PRO software (Becton-Dickinson) with a mean of 10,000 events to count % of cells positive for cell surface markers of CD90, CD73 and CD105. Other cell sorting methods can also be used; some may use antibodies to CD90, CD73 or CD105 to sort the cells.

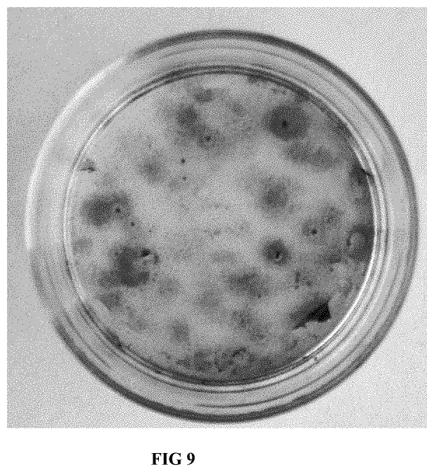

Exemplary Method of Expansion and Differentiation of SF-MSC/SF-PCs: Synovial fluid is obtained from the subjects and processed under sterile conditions in a laminar flow hood. Each sample is placed in a 50 ml polypropylene sterile tube for centrifugation at 1500 rpm for 10 minutes to obtain a pellet. The cell pellet is suspended in DMEM (Dulbecco's Minimum Essential Medium), supplemented with 20% autologous serum (filtered at 0.22 □m) or 20% Fetal Bovine Serum and 1% antibiotic-antimycotic). Cells are counted and cultured in T-12 flasks or 35 mm culture plates and incubated at 37° C. and 5% CO₂ for 48 hours. The cells are then fed every 48 hours until they reach 90-95% confluence, typically in 2-3 weeks. At the time of harvest, the synovial fluid stem cells positive for CD73, CD90 and CD105 markers (SF-MSCs/SF-PCs), comprising 0.5-3% of total population. At the end of the culture (at confluence in 2-3 weeks), this number increases to about 80-94%, with the majority of the cells fitting the characterization of Progenitor cells (SF-PCs). After plating, SF-MSCs grow vigorously, form colonies and dominate the culture (FIG. 9). Then begin to differentiate to SF-PCs and in a paracrine manner, influence the differentiation of other stem cells either by cell-to-cell communication via secretion of signaling factors, or by way of the extracellular vesicles (Exosomes) until at confluence, an almost homogeneous population of SF-PCs is achieved. The described physiologically conductive environment is adequate for differentiation of up to 80-94% SF-PCs, in the span of 2-3 weeks, without the need for any external growth factors or genetic manipulation. Other culturing methods can be used.

Exemplary Method of Formation of Cartilage Nodules in vitro: The synovial fluid-derived mesenchyme stem cells are sorted by flow cytometry and an average of 2×10⁴ cells/cm² surface area are plated as described above. The culture is fed with DMEM with 20% FBS and antibiotics every 48 hours until confluence, typically in a span of 2-3 weeks. A sub-population of SF-PCs made by a disclosed method demonstrates biosynthesis of hyaline cartilage components including glycoproteins, glycogen, collagen type II and others in the cytoplasm. These components are often excreted to form nascent cartilage nodules in the culture dish. Formation of cartilage nodules are verified by staining of the glycoprotein-containing components with Periodic acid/Schiff Reagent (PAS)-Alcian Blue, and by immunohistochemistry of collagen type II. Methods: A) PAS Reagent-Alcian Blue staining: after fixing the cells in 3% Acetic acid solution for 3 min, they are stained with 1% Alcian Blue (pH 2.5) for 20 minutes, followed by staining with 0.5% Periodic acid for 10 minutes, and Schiff reagent for another 20 min. Stained cells are rinsed and observed under microscope. B) Immunohistochemistry for collagen type II: Cells are fixed in 2% paraformaldehyde for 20 min, rinsed with PBS, then with a solution of BPS+1% albumin+0.3% Triton for 20 minutes to block non-specific binding. The blocking solution is removed and replaced with 100 μL of the primary antibody COL2A1 at a dilution of 1:500. The culture dish is incubated overnight at 4° C. After overnight incubation, the dish is rinsed 2 times with PBS+0.1% triton, then treated with 100 μL secondary antibody at a dilution of 1:1000 (anti-mouse IgG1 FITC) for 2 hours at 4° C. The dish is again washed with PBS+0.1% triton to remove excess secondary antibody; then treated with 100 μL DAPI mounting medium Vectashield (Vector cat. H-1200) in dark for 10 min. Cells are then washed one more time and observed in a microscope system with the software image Vision. Other methods can be used.

In other aspects, this disclosure provides a method of treating a subject comprising: administering a therapeutically effective number of chondroblasts, or progenitors thereof, or a pharmaceutical composition comprising the chondroblasts, or progenitors thereof, made by a disclosed method to a subject in need thereof. In certain embodiments, the subject has chondral (articular) cartilage damage, torn or damaged meniscus cartilage, torn or damaged labrum, torn or damaged joint ligament, torn or damaged patellar cartilage, or an external injury to the joint. In certain embodiments, the chondroblasts, or progenitors thereof made by a disclosed method are administered to an injured site in the subject. In further embodiments the injured site is a joint. In other embodiments, the chondroblasts, or progenitors thereof, made by a disclosed method are administered to chronically damaged tissues, in a subject in need thereof for treating osteoarthritis and the pain associated with osteoarthritis. In other embodiments, the chondroblasts, or progenitors thereof, made by a disclosed method are administered to non-injured cartilage-containing tissues, such as the tip of the nose, ear, and trachea, in a subject in need thereof for tissue reconstruction, tissue augmentation or for aesthetic reasons.

In certain embodiments, the chondroblasts, or progenitors thereof made by a disclosed method are administered by injection, use of scaffold or various matrices and glue forms, or by Carboplasty. In certain embodiments, the subject is human, racehorse or companion animal. The subject may be any animal.

In other aspects, this disclosure provides a method of treating a subject comprising: administering a therapeutically effective amount of the chondroblasts, or progenitors thereof, made by a disclosed method to a subject in need thereof.

In other aspects, this disclosure provides a method of making exosomes and/or growth/differentiating factors comprising: collecting exosomes, and/or other growth/differentiating factors, excreted by:

(i) synovial fluid stem cells or progenitor cells derived from injured or healthy joint tissues, or
(ii) mesenchyme stem cells or progenitor cells isolated from a source other than synovial fluid, wherein said source is adipose tissue or bone marrow from a mammalian subject, including a young subject; or an embryo, a fetus (including placenta and umbilical cord) or a mixture of these sources, after culturing said mesenchyme stem cells or progenitor cells for at least two weeks in vitro. The growth/differentiating factors may be collected during the collection of exosomes and may be undefined. The term “growth/differentiating factors” is used interchangeably with the term “signaling factors” herein.

In other aspects, this disclosure provides a method of differentiating allogeneic mesenchyme stem cells to SF-PCs or chondroblast cells comprising: culturing the allogeneic mesenchyme stem cells in close proximity to the exosomes or signaling factors made by a disclosed method. In some embodiments, the allogeneic mesenchyme stem cells are old, have low chondrogenic potential, or have a gender-specific sub-optimal density.

In other aspects, this disclosure provides a method of treating a subject comprising: administering the exosomes or signaling factors (or a pharmaceutical composition comprising these) made by a disclosed method to a subject in need thereof. In some embodiments, the exosomes or signaling factors made by a disclosed method are administered to an injured site in said subject. In further embodiments, the injured site is a joint. In other embodiments, the exosomes or signaling factors made by a disclosed method are administered to chronically damaged tissues in said subject for treating osteoarthritis and the pain associated with osteoarthritis. In some embodiments, the exosomes or signaling factors made by the disclosed method are administered by injection, by use of scaffold or various matrix and glue forms, or by Carboplasty. In certain embodiments, the subject is human, racehorse or companion animal.

The subject in need of exosome or signaling factor treatment may be old, have low regenerative potential, or have low stem cell density, as frequently seen in female in comparison to male subjects; in need of cartilage regeneration in the joints to treat chondral (articular) cartilage damage, torn or damaged meniscus cartilage, torn or damaged labrum, subchondral bone edema, torn or damaged joint ligament, torn or damaged patellar cartilage, or an external injury to the joint.

In some embodiments, the subject in need of exosome or signaling factor treatment is not injured but can use exosome/signaling factor treatment for tissue augmentation and aesthetic purposes. In such embodiments, the exosomes or signaling factors made by the disclosed method are derived from injured tissues of younger subjects and used for tissue augmentation of the older and/or compromised subjects in an allogeneic procedure. In other embodiment, the exosomes or signaling factors made by the disclosed method are derived from embryonic cells, or by a combination of exosomes or signaling factors from both adult and embryonic sources. In some embodiments, the exosomes or signaling factors are derived from mesenchyme stem or progenitor cells originated from sources other than the synovial fluid, including adipose tissue, bone marrow and fetal and placental tissues.

This disclosure provides a method of tissue augmentation therapy comprising: administering a therapeutically effective amount of the exosomes and/or signaling factors made by a disclosed method to a subject in need thereof. In some embodiments, said exosomes or signaling factors are administered to a non-injured site in said subject. In some embodiments, said exosomes or signaling factors are administered by injection, by use of scaffold or various matrices, mesh and glue forms. In certain embodiments, the subject is human, racehorse or companion animal.

In further embodiments, a disclosed method of treatment further results in effective treatment.

Tissue and cell culture may be performed by methods known in the art using suitable media, etc. known in the art.

The term “close proximity” as used herein refers to the cell culture density of approximately 50,000 cells per 8 cm² of growth area.

The term “young subject” as used herein refers to a subject in the first 25% of the subject's natural life.

The term “carboplasty” refers to the technique used to implant cells into the chondral-bone interface, using Pecaboo instruments (Vad Holdings, LLC).

Exemplary Method of Making Exosomes: Exosomes are isolated from culture media of SF-PCs. Approximately 48 hour prior to harvesting of exosomes, cells are treated with serum-free media to exclude presence of any serum-derived exosomes. Exosomes are isolated from the culture media by filtering through 0.22 □m pore size and, by subsequent extraction by a commercially available exosome isolation kit such as the Genexosome Technologies GET™ Exosome isolation kit (Cat #:GET301-10). The extracted exosomes are kept in −80° C. until used. To check for presence of exosomes, western blot analysis of exosome protein extracts, or transmission electron microscopy are used. Progenitor cell-derived exosomes express several surface proteins of the MSCs/PCs including CD90 and CD73. Anti CD90 antibody is used to validate and quantify the secreted exosomes by western blot analysis. To measure exosomes quantity, Nanoparticle Tracking machines such as NanoSight NS300 are used. Other extraction methods can be used. Signaling factors may also be obtained in substantially the same way and by biochemical analytical techniques.

The immunological inertness of the extracted exosomes may be ascertained.

Exemplary Method of Making Chondroblasts by Exposure to Allogeneic Exosomes: The isolated exosomes are used to promote growth and/or differentiation of allogeneic SF-MSC populations. Exosomes are extracted from the culture media of SF-PCs of an injured (or healthy) younger patient by a disclosed method, and stored at −80° C. SF-MSCs harvested from an older patient are made by a disclosed method and treated with culture media containing said allogeneic exosomes. The cells are treated with exosomes every 48 hours until they reach confluence, then tested for the presence of collagen type II and cartilage in vitro, to validate chondrogenesis.

In some embodiments, a disclosed method for treating a subject is for treating chondral (articular) cartilage damage by cartilage regeneration in the joints, torn or damaged meniscus cartilage, torn or damaged labrum, subchondral bone edema, torn or damaged joint ligament, torn or damaged patellar cartilage, or an external injury to the joint in the subject.

Pharmaceutical Compositions and Formulations

Compositions containing the chondrocytes or progenitors thereof made by the disclosed methods or the exosomes or signaling factors made by the disclosed methods can be formulated as a pharmaceutical composition for administering to a subject. Any suitable pharmaceutical compositions and formulations, as well as suitable methods for formulating and suitable routes and suitable sites of administration, are within the scope of this disclosure. Also, unless otherwise stated, any suitable dosage(s) and frequency of administration are contemplated.

The pharmaceutical compositions can include a pharmaceutically acceptable carrier (i.e., an excipient). A “pharmaceutically acceptable carrier” refers to, and includes any and all solvents, dispersion media, coatings, antibacterial and antifungal agents, isotonic and absorption delaying agents, diluent, glidant, etc. The compositions can include a pharmaceutically acceptable salt, e.g., an acid addition salt or a base addition salt (see e.g., Berge et al. (1977) J Pharm Sci 66:1-19). The composition can be coated when appropriate.

Any suitable route of administering the chondrocytes, or progenitors thereof, made by a disclosed method or exosomes or signaling factors, made by a disclosed method, may be used. In certain embodiments, the chondrocytes, or progenitors thereof, made by a disclosed method or exosomes made by a disclosed method, or pharmaceutical composition comprising either, are administered by injection, scaffold or various matrix and glue forms, or by Carboplasty.

Exemplary Method of Implantation of SF-MSCs or SF-PCs and/or Exosomes: synovial fluid derived stem/progenitor cells and/or the extracted exosomes and/or signaling factors are implanted using the following methods, or comparable implantation techniques, and will be checked for outcomes on months 1.5, 3, 6 and 12 post-implantation. Mini Koos pain scale and Pre- and post MRI scanning for edema reduction and CartiGram (T2 mapping sequence) are used to map increase in cartilage thickness. Other implantation methods can be used.

Arthroscopic Implantation with Scaffolding: Autologous SF-MSCs are grown in vitro until confluence is reached (2-3 weeks), as described before. Ensuing SF-PCs are detached from flasks by the use of Trypsin-EDTA for 5 minutes. The enzyme solution is neutralized by growth medium and the cell suspension is transferred to a 15 mL tube for centrifugation for 10 minutes at 1500 rpm. The cells are subjected to cytometry to assure the presence of at least 85% SF-PCs. Combi-set II (Thrombin) is prepared according to the kit instructions and 300 □l of it is mixed with an equal volume of cell suspension including 2.5-3 million cells. The mixture is then seeded on a piece of Hyalofast scaffolding, cut to fit the cartilage deficiency of the patient. Combi-Set I (Fibronectin) is also prepared according to the kit instructions and poured over seeded scaffolding to create a seal. The ensemble is then allowed 10 minutes to set, placed in a petri dish and covered with DMEM and 20% autologous (or fetal bovine) serum. The culture dish including the seeded scaffolding is incubated at 37° C. and 5% CO² for 3 days before implantation.

At the day of surgery, the subject is treated with epidural anesthesia, an arthroscope is inserted to view the site of injury and a cannula is inserted to guide the scaffolding to the site. The cartilage site is cleared of damaged tissue and debris until clear margins are attained. The seeded scaffolding is trimmed of the excess fibrin glue. With the help of an arthroscopic clamp, the scaffolding is introduced to the injury site through the cannula. Fibrin glue is introduced to the implantation site and the scaffolding is held under light pressure until firmly in place. More fibrin glue is added to the implantation site to assure adherence.

Other implantation methods can be used.

Percutaneous Delivery of SF-MSCs or SF-PCs and/or Exosomes to chondral-bone Interface: Patients with unicompartmental knee Osteoarthritis who have failed conservative care are used. 2-3 ml autologous chondroblast cell suspension (with/or without exosomes and/or signaling factors) is mixed with equal volume of calcium gluconate and injected (via PeCaBoo-Vad Holdings, LLC) into the superior and inferior chondral-bone interface. The same volume of suspension is also injected intra-articularly. Other delivery methods can be used.

### EXAMPLES

For this invention to be better understood, the following examples are set forth. These examples are for purposes of illustration only and are not be construed as limiting the scope of the invention in any manner.

### Example 1—Formation of Cartilage by SF-PCs In Vitro: 27 Year Old Male with Medial Femoral Condyle Grade IV Knee Injury

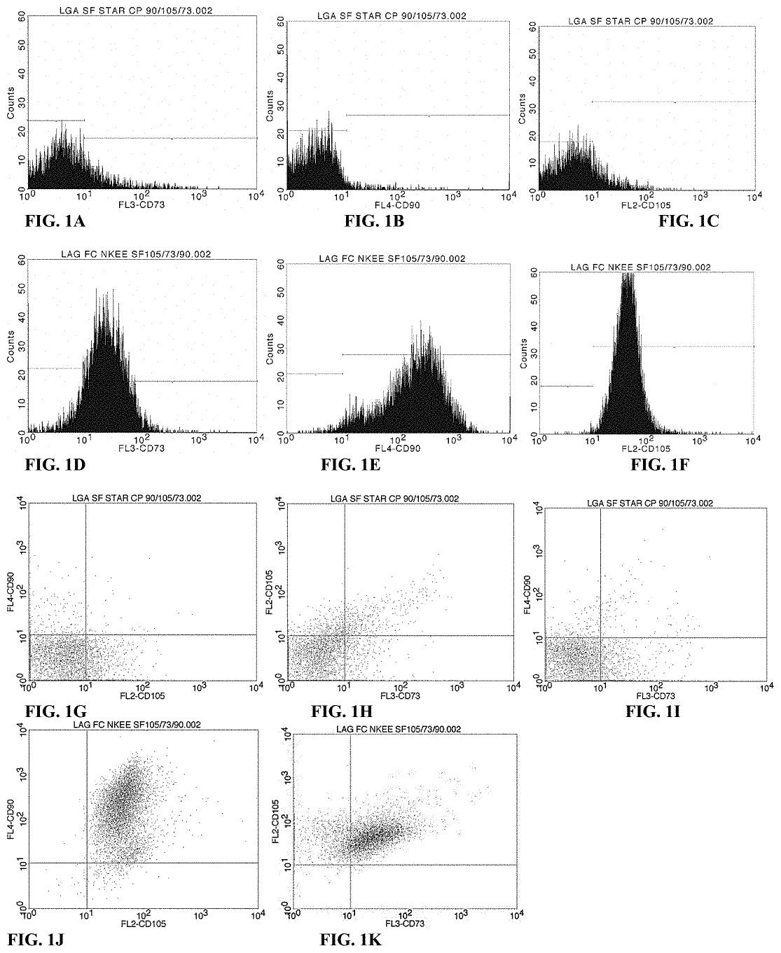

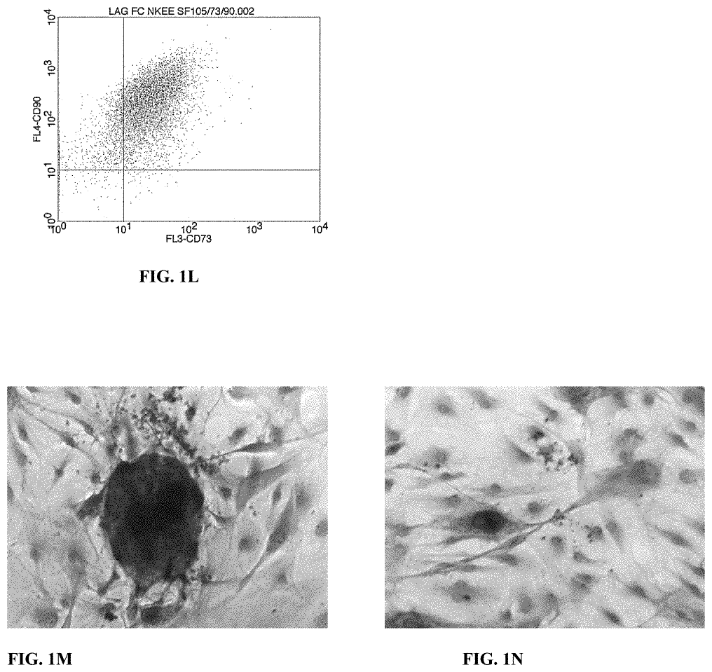

Synovial Fluid irrigation of this patient's injured knee was performed under regional anesthesia—10 million SF-MSCs/SF-PCs with viability of 80% were harvested. Cells were spun and counted, then plated in a T-25 flask in DMEM and 20% autologous serum. Growth medium was replaced every 48 hours until confluence was reached, 16 days later. At that time, cells were stained with PAS/Alcian blue for presence of cartilage and glycosaminoglycans (FIG. 1M-FIG. 1N). Cells were also subjected to flow cytometry to identify mesenchyme stem cells positive for surface markers CD73, CD90 and CD105 at the time of harvest (FIG. 1A-FIG. 1C and FIG. 1G-FIG. 1I) and chondrogenic, or Progenitors thereof, at confluence (FIG. 1D-FIG. 1F and FIG. 1J-FIG. 1L).

CD analysis of SF-MSC populations indicated significant increase of the number of SF-PCs with increasing chondrogenic profile from day 1 to confluence (day 16).

### Example 2—Formation of Cartilage by SF-PCs In Vitro: 22 Year Old Male with Anterior Cruciate Ligament Injury, and Meniscus Lesion

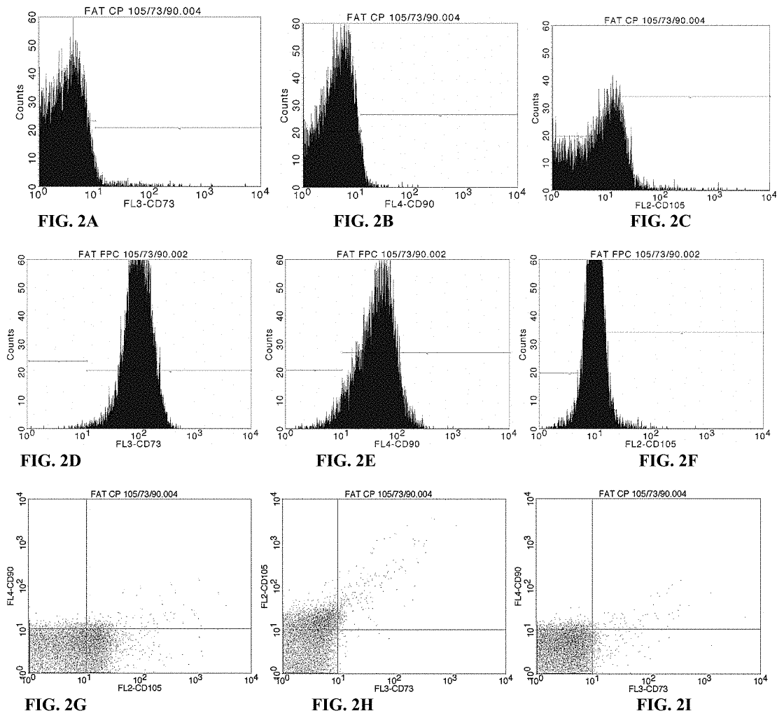

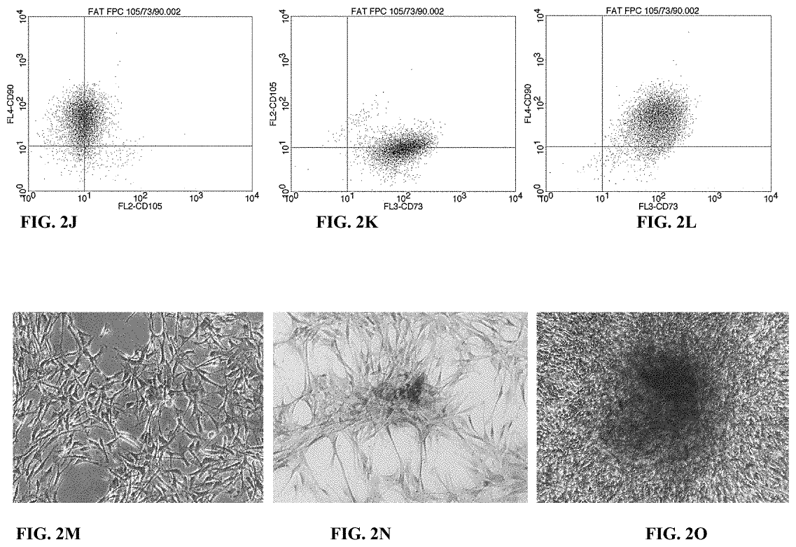

Synovial Fluid irrigation was performed of this patient's injured site, using 30 ml PBS—1.49M cells with viability of 90% were harvested. Cells were spun and counted, then plated in a T-25 flask in DMEM and 20% autologous serum. Growth media were replaced every 48 hours until confluence 15 days later. At that time cells were stained with PAS for presence of cartilage (FIG. 2M-FIG. 2O). Cells were also subjected to flow cytometry to identify stem cell surface markers CD73, CD90 and CD105 at the time of harvest and chondrogenic/Progenitor at confluence on Day 15 (FIG. 2D-FIG. 2F and FIG. 2J-FIG. 2L).

CD analysis of SF-MSC populations indicates significant statistical increase of the number of SF-PCs with positive chondrogenic profile from day 1 to confluence (day 15).

### Example 3—Chondrogenic Potential of Injured Versus Healthy SF-MSCs

32 year old female with injuries to the left knee (Chondral lesion grade IV on the lateral side and medial patella) was presented. Synovial fluid irrigation of her left knee was performed under regional anesthesia using 30 ml PBS-540K SF-MSCs/SF-PCs with viability of 80% were harvested. At the same time an exploratory arthroscopy was performed on the (right/uninjured) knee to examine the cause of patellar hyper-pressure syndrome. Synovial fluid was collected from the healthy (right) knee and used for comparison with the injured (left) knee. Cells were spun and counted, then plated in T-25 flasks in DMEM and 20% serum. Growth media were replaced every 48 hours until confluence was achieved. At that time, cells were stained with PAS/Alcian blue for presence of cartilage. Cells were also subjected to flow cytometry to identify mesenchyme stem cell surface markers CD73, CD90 and CD105 at the time of harvest, and SF-PCs at confluence.

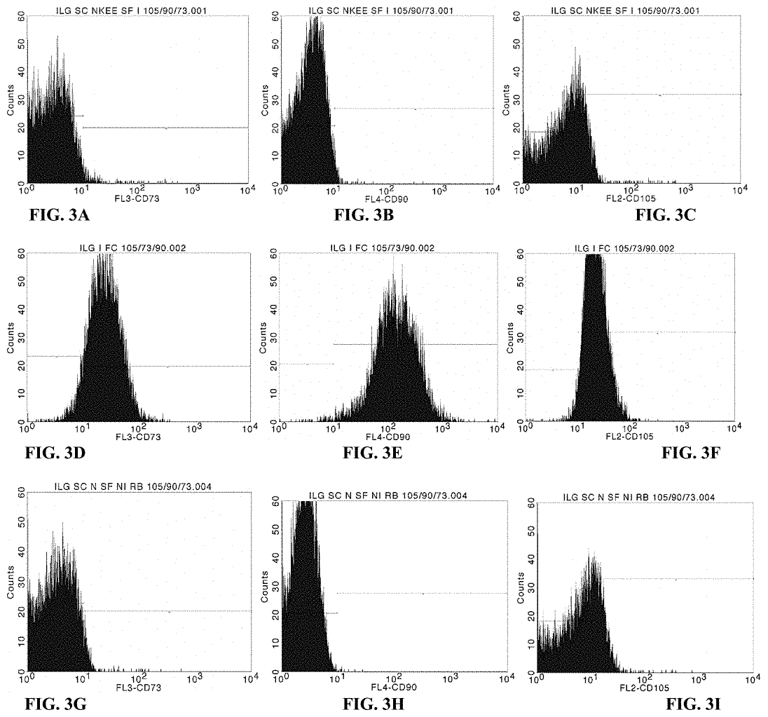

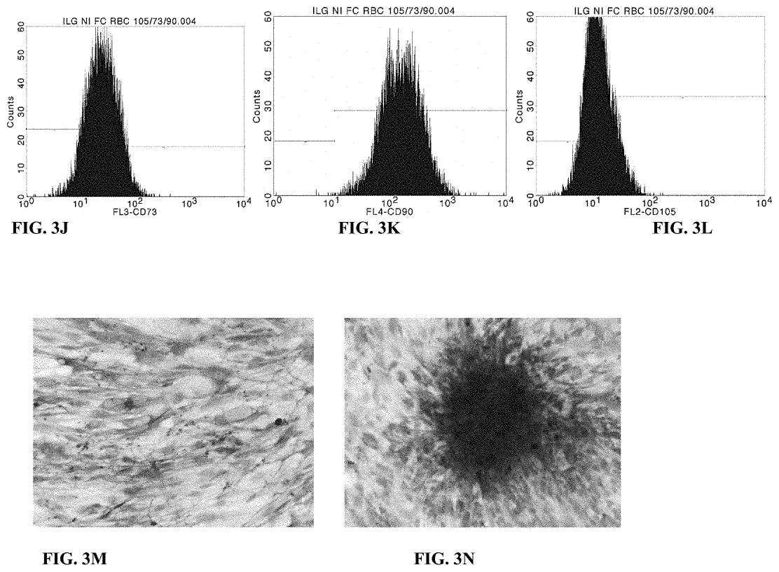

Although flow cytometry profile of SF-PCs derived from healthy (FIG. 3G-FIG. 3L) and injured (FIG. 3A-FIG. 3F) knees were not statistically different, PAS staining showed higher presence of cartilaginous nodules in the culture of SF-PCs derived from the injured knee (FIG. 3N) vs. the healthy knee (FIG. 3M).

### Example 4—Implantation of SF-PCs Using Scaffolding

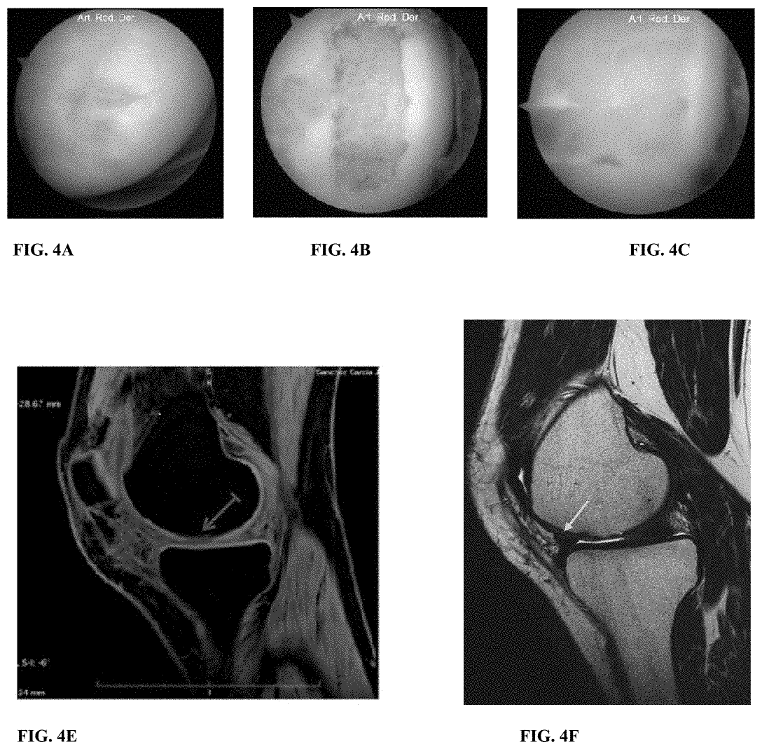

37 year old female with lateral femoral trochlear chondral focal lesion, grade IV (FIG. 4A), underwent focal cartilage repair using autologous SF-PCs-seeded Hyalofast scaffolding. Autologous SF-MSCs/SF-PCs from the injured site were harvested and grown until confluence, as per the disclosed method. Cells were detached and infused in a section of hyalofast tailored to the deficient area for 3 days before implantation.

On the day of surgery, the damaged cartilage was cleared of debris until clear margins were attained (FIG. 4B). The seeded scaffolding was introduced to the injury site through a cannula. Fibrin glue was added to the implantation site and held under light pressure until firmly in place (FIG. 4C).

4 weeks post implantation imaging showed adherence of the implant and progression of healing. (FIG. 4D). 12 month post-implantation Mill showed regeneration of cartilage (FIG. 4E).

### Example 5—Exosome Farming and Quantification

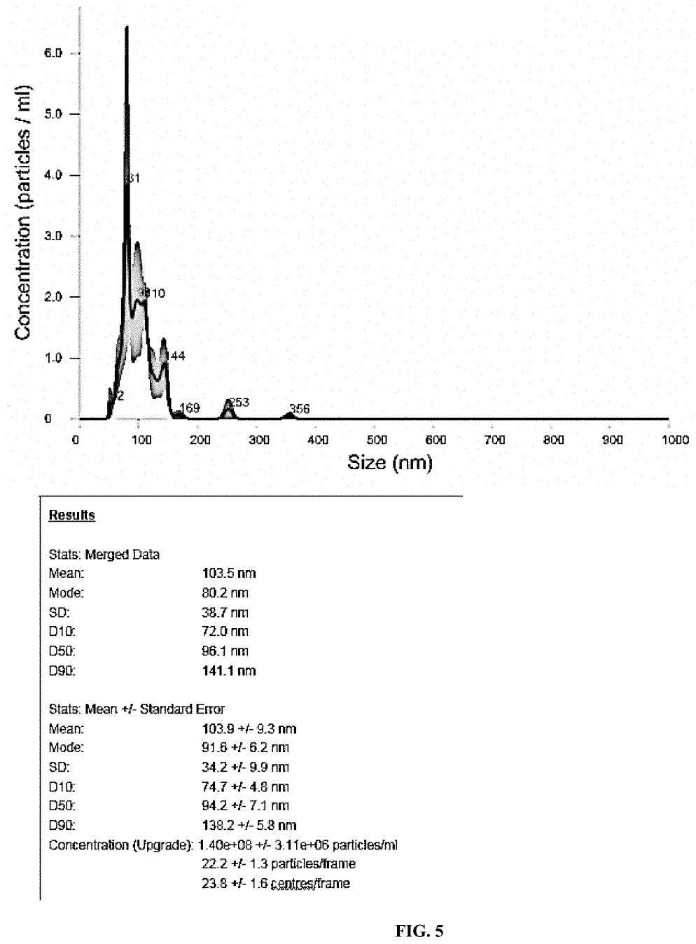

A suspension of 1×10⁵ passage 2 Triple positive (CD90⁺CD105⁺CD73⁺) SF-MSC/SF-PCs of a 54 year old female, were cultured in T-12 flasks and incubated at 37° C. and 5% CO₂. The culture was fed every 48 hours until confluence was reached. 72 hours prior to the harvest, the cells were grown in serum-free DMEM to exclude exosomes originated from serum. SF-PC-derived exosomes were isolated from culture media using Qiagen exoEasy Maxi Kit and measured on Malvern Nanosight machine. Analysis measured 2.8×10¹⁰/ml exosomes with average diameter of 80.2 nm (FIG. 5)

### Example 6—Therapeutic Application of Exosomes and/or Signaling Factors

77 year old female was presented with decreased joint space, marginal osteophytes, and damaged articular cartilage and meniscus. The patient underwent total knee replacement surgery. During the procedure, synovial fluid was collected from the injured knee—510,000 SF-MSCs/SF-PCs with 90% viability were harvested. Cells were spun and counted, then plated in duplicates in 12-well plates as per the following method: 10,000 cells in 0.5 ml DMEM and 20% autologous serum; and 10,000 cells in 0.5 ml 0.22 □m filtered serum-deprived cell culture supernatant from Day 16 (confluence state) of a younger subject (22 year old), including exosomes and/or signaling factors. Cultures were allowed to grow for 21 days. Growth and differentiation of the cells treated with patient's serum versus cells treated with supernatant from the younger patient (containing exosomes and/or signaling factors) were compared to demonstrate induction of differentiation of the SF-MSCs/SF-PCs of the older patient by allogeneic exosomes and/or signaling factors from the younger patient.

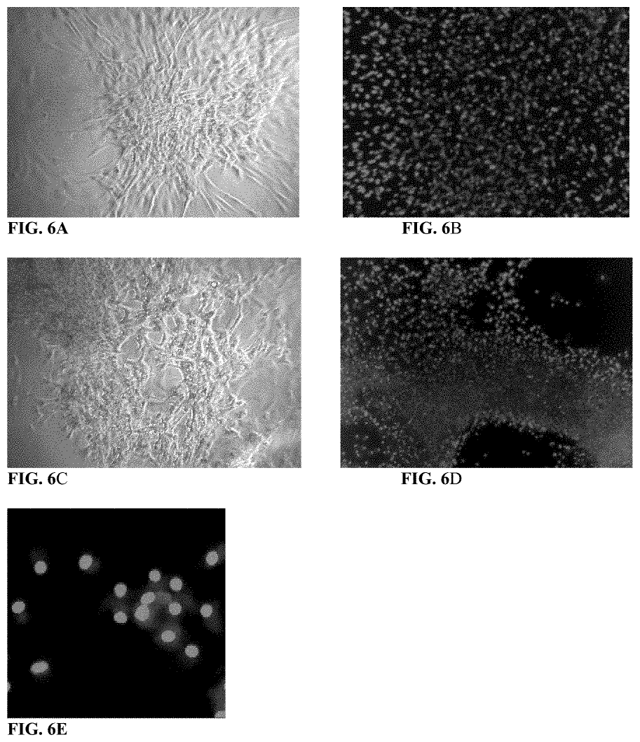

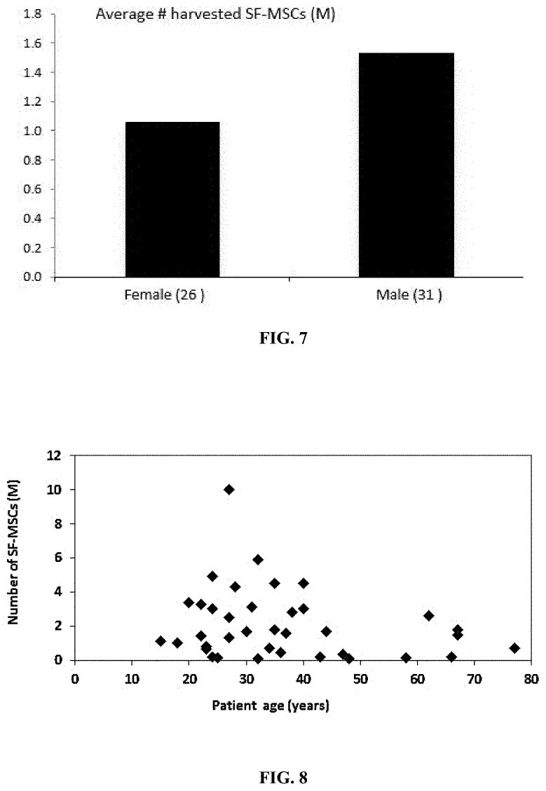

The group treated with autologous serum showed higher number of cells (FIG. 6A), however, the cells appeared fibro-cartilage like; while the cell in group treated with exosomes and/or signaling factors of the younger patient, were more differentiated and showed evidence of formation of cartilage components (FIG. 6C). Immunohistochemistry staining for Collagen type II showed no production in the autologous serum-treated cultures (FIG. 6B), while the group treated with exosomes and/or signaling factors of the younger patient demonstrated higher expression of collagen type II in the cytoplasm of the cells (FIG. 6D and FIG. 6E), further confirming the formation of cartilage by the treated group.

### Example 7—Influence of Gender and Age on Cartilage Regenerative Ability

Using methods disclosed herein, the following observations are made.

The average number of SF-MSCs/SF-PCs isolated from 31 male patients was higher than 26 female subjects, both at harvest and at confluence (1.49 million vs 1.01 million at harvest, and 1.30 million vs 0.69 million at confluence; p<0.05) (FIG. 7). The average growth rate of SF-MSCs harvested from male patients was also higher than female subjects (20.21 days vs 24.56 days)—Although not statistically significant, it trended toward significance (Mann-Whitney p=0.079).

A progressive decline in the number of harvested SF-MSCs/SF-PCs in synovial fluid of patients from the age 18 to age 79 was also observed (FIG. 8).

### Additional Aspects and Embodiments

This disclosure provides a method of making mammalian chondroblast cells, or progenitors thereof, comprising: growing free-floating synovial fluid cells extracted from a subject's synovial cavity resulting in proliferation and formation of an adherent cell culture containing one or more colonies of mesenchymal stem cells exhibiting CD90, CD73 and CD105 cell surface markers, wherein said colonies of the stem cells constitute at least about 88% of total cells in said cell culture, wherein said growing step is performed without any added growth and differentiation factors. In some embodiments, said growing step is performed without using a 3-D scaffold, or layering of extracted cells with other cell types. In some embodiments, said growing step is performed without separating different cell types in said synovial fluid derived free-floating cells. In some embodiments, the method further comprises, subsequent to the extraction of the synovial fluid and prior to said growing step, determining a percentage of the stem cells exhibiting said CD90, CD73 and CD105 surface markers in said synovial fluid. In some embodiments, a temporal duration of the growing step is in a range of about 2 weeks to about 3 weeks. In some embodiments, said synovial fluid is extracted from a synovial cavity of a subject with an injured joint. In some embodiments, said synovial fluid is extracted from a synovial cavity of a subject with a healthy joint. In some embodiments, the subject is a human, a horse, or a companion animal.

This disclosure also provides a method of treating a subject comprising: administering a therapeutically effective number of chondroblast cells, or progenitors thereof, to a subject in need thereof, wherein said chondroblast cells, or progenitors thereof are made via differentiation of a plurality of stem cells made by growing free-floating synovial fluid cells extracted from a synovial cavity so as to allow proliferation and formation of an adherent cell culture containing one or more colonies of mesenchymal stem cells exhibiting CD90, CD73 and CD105 cell surface markers, wherein said colonies of the stem cells constitute at least about 88% of total cells in said cell culture, and wherein said growing step is performed without any added growth or differentiation factors. In some embodiments, the growing step is performed over a period of about 2 to about 3 weeks. In some embodiments, the growing step is performed without separating different cell types in said synovial fluid derived free-floating cells. In some embodiments, said chondroblast cells, or progenitors thereof, are administered to an injured site in said subject. In some embodiments, the injured site is a joint. In some embodiments, said chondroblast cells, or progenitors thereof, are administered to a chronically damaged tissue in said subject for treating osteoarthritis and pain associated with osteoarthritis. In some embodiments, the method is for treating chondral (articular) cartilage damage by cartilage regeneration in a joint, torn or damaged meniscus cartilage, torn or damaged labrum, subchondral bone edema, torn or damaged joint ligament, torn or damaged patellar cartilage, or an external injury to the joint in said subject. In some embodiments, said chondroblast cells, or progenitors thereof, are administered to a non-injured cartilage-containing tissue in said subject for tissue reconstruction, tissue augmentation or for aesthetic reasons. In some embodiments, said cartilage-containing tissue comprises tip of the nose, ear, or trachea. In some embodiments, said chondroblast cells, or progenitors thereof, are administered by any of injection, a scaffold, a matrix and a glue, and Carboplasty. In some embodiments, said subject is human, horse or companion animal.

This disclosure also provides a method of making extracellular vesicles comprising: collecting extracellular vesicles excreted by any of:

synovial fluid mesenchyme stem cells (SF-MSCs) harvested from injured or healthy joint tissues; or chondroblast cells, or progenitors thereof, made from said synovial fluid mesenchyme stem cells (SF-MSCs); or mesenchyme stem cells or progenitor cells isolated from a source other than synovial fluid;
wherein the chondroblast cells, or progenitors thereof, are made by a method comprising: harvesting free-floating synovial fluid cells from a subject's synovial cavity; allowing proliferation and formation of an adherent cell culture containing one or more colonies of mesenchymal stem cells exhibiting CD90, CD73 and CD105 cell surface markers, wherein said colonies of the stem cells constitute at least about 88% of total cells in said cell culture, wherein, the temporal duration of the growing step is in a range of about 2 weeks to about 3 weeks; and collecting a plurality of extracellular vesicles from culture medium of said cell culture. In some embodiments, the proliferation and formation of an adherent cell culture (i.e., growing) step is performed without any added growth and differentiation factors, and/or use of a 3-D scaffold, and/or layering of extracted cells with other cell types. In some embodiments, the subject is a human, a horse, or a companion animal. In some embodiments, the source of mesenchyme stem cells other than the synovial fluid comprises any of adipose tissue, bone marrow from a mammalian subject, an embryo, a fetus, placenta, umbilical cord and mixtures thereof after culturing said mesenchyme stem cells or progenitor cells thereof for at least two weeks in vitro. In some embodiments, the collected extracellular vesicles have a diameter in a range between about 150 and about 300 nm. In some embodiments, the collected extracellular vesicles have a diameter in a range of about 100 nm to about 600 nm. In some embodiments, the extracellular vesicles comprise exosomes.

This disclosure provides a method of differentiating mesenchyme stem cells of a recipient subject to progenitor chondroblast cells, in vivo or in vitro, comprising: exposing said mesenchyme stem cells to a plurality of extracellular vesicles made by a disclosed method, derived from any of stem cells, or progenitors chondroblast cells thereof, derived from an allogeneic donor. In some embodiments, the recipient subject is in the last trimester of a normal range of life expectancy. In some embodiments, the recipient subject has low chondrogenic potential. In some embodiments, the low chondrogenic potential is gender-related.

This disclosure also provides a method of autologous or allogeneic treatment of cartilage damage so as to facilitate cartilage regeneration of a recipient, comprising: administering a therapeutically effective amount of the extracellular vesicles made by a disclosed method to a subject in need of said treatment. In some embodiments, the extracellular vesicles are converted to a lyophilized form prior to said administration. Lyophilization can be done by any method known in the art. In some embodiments, the therapeutically effective amount of the extracellular vesicles is substantially cell-free. In some embodiments, the extracellular vesicles are administered for joint therapy. In some embodiments, the extracellular vesicles are administered to a chronically damaged tissue in said subject for treating osteoarthritis and pain associated with osteoarthritis. In some embodiment, the extracellular vesicles are administered by any of injection, a scaffold, a matrix and a glue, and Carboplasty. In some embodiments, the method is for treating chondral (articular) cartilage damage by cartilage regeneration in a joint, torn or damaged meniscus cartilage, torn or damaged labrum, subchondral bone edema, torn or damaged joint ligament, torn or damaged patellar cartilage, or an external injury to the joint in said subject. In some embodiments, the extracellular vesicles are administered to a non-injured site in said subject for tissue reconstruction or aesthetic purposes. In some embodiments, the subject is a human, a horse, or a companion animal. In some embodiments, the extracellular vesicles comprise exosomes.

In some embodiments, a therapeutically effective combination of extracellular vesicles (e.g., exosomes) and mesenchyme stem cells disclosed herein can be administered to a chronically damaged tissue in a subject for treating osteoarthritis and pain associated with osteoarthritis. In some embodiments, such a combination of extracellular vesicles and mesenchyme stem cell are administered by any of injection, a scaffold, a matrix and a glue, and Carboplasty. In some embodiments, the method is for treating chondral (articular) cartilage damage by cartilage regeneration in a joint, torn or damaged meniscus cartilage, torn or damaged labrum, subchondral bone edema, torn or damaged joint ligament, torn or damaged patellar cartilage, or an external injury to the joint in said subject. In some embodiments, a combination of the extracellular vesicles and the mesenchyme stem cells are administered to a non-injured site in said subject for tissue reconstruction or aesthetic purposes. In some embodiments, the subject is a human, a horse, or a companion animal. In some cases, the combination of the extracellular vesicles and the mesenchyme stem cells can provide a synergistically enhanced treatment results.

This disclosure provides a method of making extracellular vesicles including exosomes, comprising: collecting extracellular vesicles excreted by any of (i) synovial fluid mesenchyme stem cells (SF-MSCs) or chondroblast cells, or progenitors thereof from injured or healthy joint tissues of a subject, and (ii) mesenchyme stem cells or progenitor cells isolated from a source other than synovial fluid of a subject, wherein the step of collecting extracellular vesicles comprises: growing adherent cell culture containing mesenchymal stem cells exhibiting CD90, CD73 and CD105 cell surface markers for at least two passages, and subsequently, collecting exosomes from culture medium of said cell cultures. In some embodiments, the source of mesenchyme stem cells is other than the synovial fluid comprises any of adipose tissue, bone marrow from a mammalian subject, an embryo, a fetus, placenta, umbilical cord and mixtures thereof after culturing said mesenchyme stem cells or progenitor cells thereof, for at least two weeks in vitro. In some embodiments, said collected extracellular vesicles have a diameter in a range between about 150 and about 300 nm. In some embodiments, said collected extracellular vesicles have a diameter in a range of about 100 nm to about 600 nm. In some embodiments, the extracellular vesicles comprise exosomes.

This disclosure provides a method of differentiating mesenchyme stem cells of a recipient subject to progenitor chondroblast cells, in vivo or in vitro, comprising: exposing said mesenchyme stem cells to a plurality of extracellular vesicles derived from stem cells or progenitors chondroblast cells thereof made by a disclosed method derived from an allogeneic donor. In some embodiments, said recipient subject is old. In some embodiments, said recipient subject has low chondrogenic potential. In further embodiments, said low chondrogenic potential is gender-related.

This disclosure provides a method of autologous or allogeneic treatment of cartilage damage so as to facilitate cartilage regeneration of a recipient, comprising: administering a therapeutically effective amount of extracellular vesicles made by a disclosed method to a subject in need of said treatment. In some embodiments, said extracellular vesicles are converted to a lyophilized form prior to said administration. Lyophilization can be done by any method known in the art. In some embodiments, said therapeutically effective amount of the extracellular vesicles is substantially cell-free. In some embodiments, said extracellular vesicles are administered for joint therapy. In some embodiments, said extracellular vesicles are administered to a chronically damaged tissue in said subject for treating osteoarthritis and pain associated with osteoarthritis. In some embodiments, said extracellular vesicles are administered by any of injection, a scaffold, a matrix and a glue, and Carboplasty. In some embodiments, the method is for treating chondral (articular) cartilage damage by cartilage regeneration in an a joint, torn or damaged meniscus cartilage, torn or damaged labrum, subchondral bone edema, torn or damaged joint ligament, torn or damaged patellar cartilage, or an external injury to the joint in said subject. In some embodiments, said extracellular vesicles are administered to a non-injured site in said subject for tissue reconstruction or aesthetic purposes. In some embodiments, the subject is a human, a horse, or a companion animal. In some embodiments, the extracellular vehicles comprise exosomes.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A method of making mammalian chondroblast cells, or
progenitors thereof, comprising:
growing free-floating synovial fluid cells extracted from a subject's synovial cavity, resulting in proliferation and formation of an adherent cell culture containing one or more colonies of mesenchymal stem cells exhibiting CD90, CD73 and CD105 cell surface markers, wherein said colonies of the stem cells constitute at least about 88% of total cells in said cell culture;
wherein said growing step is performed without any added growth and differentiation factors.

2-27. (canceled)

28. A method of making extracellular vesicles comprising:
collecting extracellular vesicles by any of:
synovial fluid mesenchyme stem cells (SF-MSCs) harvested from injured or healthy joint tissues; or
chondroblast cells, or progenitors thereof, made from said synovial fluid mesenchyme stem cells (SF-MSCs); or
mesenchyme stem cells or progenitor cells isolated from a source other than synovial fluid;
wherein said chondroblast cells, or progenitors thereof, are made by a method comprising: harvesting free-floating synovial fluid cells from a subject's synovial cavity;
allowing proliferation and formation of an adherent cell culture containing one or more colonies of mesenchymal stem cells exhibiting CD90, CD73 and CD105 cell surface markers, wherein said colonies of the stem cells constitute at least about 88% of total cells in said cell culture, wherein the temporal duration of the proliferation and formation of an adherent cell culture is in a range of about 2 weeks to about 3 weeks.

29. The method of claim 28, wherein said proliferation and formation of an adherent cell culture step is performed without any added growth and differentiation factors or use of a 3-D scaffold, and layering of extracted cells with other cell types.

30. The method of claim 28, wherein the subject is a human, a horse, or a companion animal.

31. The method of claim 28, wherein said source of mesenchyme stem cells other than the synovial fluid is selected from the group consisting of adipose tissue, bone marrow from a mammalian subject, an embryo, a fetus, placenta, umbilical cord, or mixtures thereof, after culturing said mesenchyme stem cells or progenitor cells thereof for at least two weeks in vitro.

32. The method of claim 28, wherein said collected extracellular vesicles have a diameter in a range between about 150 and about 300 nm.

33. The method of claim 28, wherein said collected extracellular vesicles have a diameter in a range of about 100 nm to about 600 nm.

34. The method of claim 28, wherein said extracellular vesicles comprise exosomes.

35. A method of differentiating mesenchyme stem cells of a recipient subject to progenitor chondroblast cells, in vivo or in vitro, comprising: exposing said mesenchyme stem cells to a plurality of extracellular vesicles made by a method of claim 28, derived from any of stem cells, or progenitors chondroblast cells thereof, derived from an allogeneic donor.

36. The method of claim 35, wherein said recipient subject is in the last trimester of a normal range of life expectancy.

37. The method of claim 35, wherein said recipient subject has low chondrogenic potential.

38. The method of claim 37, wherein said low chondrogenic potential is gender-related.

39. A method of autologous or allogeneic treatment of cartilage damage so as to facilitate cartilage regeneration of a recipient, comprising:
administering a therapeutically effective amount of extracellular vesicles made by a method of claim 28 to a subject in need of said treatment.

40. The method of claim 39, wherein said extracellular vesicles are converted to a lyophilized form prior to said administration.

41. The method of claim 39, wherein said therapeutically effective amount of the extracellular vesicles is substantially cell-free.

42. The method of claim 39, wherein said extracellular vesicles are administered for joint therapy.

43. The method of claim 39, wherein said extracellular vesicles are administered to a chronically damaged tissue in said subject for treating osteoarthritis and pain associated with osteoarthritis.

44. The method of claim 39, wherein said extracellular vesicles are administered by any of injection, a scaffold, a matrix and a glue, and Carboplasty.

45. The method of claim 39, wherein the method is for treating chondral (articular) cartilage damage by cartilage regeneration in a joint, torn or damaged meniscus cartilage, torn or damaged labrum, subchondral bone edema, torn or damaged joint ligament, torn or damaged patellar cartilage, or an external injury to the joint in said subject.

46. The method of claim 39, wherein said extracellular vesicles are administered to a non-injured site in said subject for tissue reconstruction or aesthetic purposes.

47. The method of claim 39, wherein the subject is a human, a horse, or a companion animal.
